(19) United States
(12) Patent Application Publication
(10) Pub. No.: US 2004/0241036 A1
(43) Pub. Date: Dec. 2, 2004

(54) Medical implant for the human or animal body

(76) Inventors: Andrea Meyer-Lindenberg, Lehrte (DE); Henning Windhugen, Hannover (DE); Frabnk Witte, Hannover (DE)

Correspondence Address: Lichti Lempert & Lasch, Bergwaldstr. 1, Karlsruhe, D-76227, DE

(21) Appl. No.: 10/479,601

(22) PCT Filed: Jun. 11, 2002
(86) PCT No.: PCT/EP02/06375

(30) Foreign Application Priority Data
Jun. 11, 2001 (DE) 101 28 100.5

(51) Int. Cl. C22C 23/06; A61F 2/28

(52) U.S. Cl. 420/405; 606/76; 623/23.53

(57) ABSTRACT

A medical implant for the body of a person or an animal consists at least partially of a magnesium alloy. The magnesium alloy contains portions of rare earth metals and lithium and optionally yttrium and aluminum. The magnesium alloy preferably contains lithium in a portion of 0.01 to 7 mass %, aluminum in a portion of 0.01 to 16 mass %, optionally yttrium in a portion of 0.01 to 7 mass %, and rare earth metals in a portion of 0.01 to 8 mass %.

The invention concerns a medical implant for the body of a person or an animal which is at least partially made from a magnesium alloy.

There are various conventional medical implants of this type. They may be mounting elements for a bone (e.g. plates, screws, or nails), surgical stitching material, surgical fabric or foils or prostheses or prosthesis parts. The currently used implants are generally made from corrosion-resistant material such as special steel or titanium. Such implants disadvantageously fail to degrade in the body and must be surgically removed when they are no longer medically required, since they would eventually be rejected by the body. Alternatively, degradable implants of polymers are also known. They have, however, relatively poor strength and ductility.

As has been known since the start of the 20^{th} century, implants made from magnesium or magnesium alloys have certain advantages, since magnesium is easily degradable. The article “Magnesium Screw and Nail Transfixion in Fractures” by Earl D. McBride in “Southern Medical Journal, 1938, Vol. 31, No. 5, pages 508 ff describes the use of screws, bolts and dowels of magnesium or magnesium alloys. DE 197 31 021 A1 also discusses these issues but without mentioning the conventional degradable implants of magnesium or magnesium alloys. The magnesium alloys described in connection with bone surgery have the disadvantage of producing a relatively large gas volume per unit time, in particular hydrogen gas, which can cause gas pockets in the body having the implant which, in turn, impede the healing process since, in particular, such gas pockets cause separation of tissues and tissue layers. Moreover, the known magnesium alloys have non-uniform corrosion, which does not ensure reliable strength during the required healing time.

Surgical stitching material of magnesium or magnesium alloys has been known for a long time and is described e.g. in DE 630 061, DE 676 059, DE 665 836 and DE 688 616. A stitching material of this type also has the above-mentioned disadvantages of gas generation and non-uniform corrosion.

Magnesium or magnesium alloys have also been vacuum-evaporated onto implants, in particular of special steel, since these materials contribute to fast recovery of the bone. Prostheses or prosthesis parts which consist of corresponding material have also been used. To promote bone growth, calcium and cadmium may be added to the alloys. In addition to the above-mentioned disadvantages, the use of cadmium (Cd) is particularly problematic, since it is a toxic metal which should not enter into the body tissue.

It is therefore the underlying purpose of the present invention to provide a medical implant for the body of a person or an animal, which avoids the above-mentioned disadvantages and which can be degraded by the body with no or only minor side effects.

This object is achieved in accordance with the invention with a medical implant of the above-mentioned type in that the magnesium alloy contains rare earth metals and lithium. The rare earth metal portion contained in the magnesium alloy takes up the hydrogen produced during corrosion of the magnesium. The admixture of the rare earth metals to the magnesium alloy leads to grain refinement, producing slow, continuous and well-controlled corrosion development in the associated body implant. In this fashion, excessive gas development and the risk that gas pockets form during degradation of the implant are reliably prevented. The lithium increases the number of cover layer components and leads to very good corrosion protection for the magnesium alloy.

The addition of rare earth metals to magnesium-based alloys also improves their mechanical material properties. The inventive degradable alloy is characterized by increased ductility and increased strength accompanied by good corrosion resistance compared to the conventional degradable magnesium alloys for implants.

In accordance with the invention, the rare earth metals used are preferably cerium and/or neodymium and/or praseodymium or another element having an atomic number of 57 to 71 of the periodic system. Cerium is preferred since it is a natural component of the body and, in particular, of the bone.

In a preferred embodiment of the invention, the magnesium alloy contains:

Lithium in a portion of at least 0.01 mass % and up to 7 mass %;

Aluminum in a portion of at least 0.01 mass % and up to 16 mass %

Optionally yttrium in a portion of at least 0.01 mass % up to 7 mass % and

Rare earth metals in a portion of at least 0.01 mass % and up to 8 mass %.

The magnesium alloy is preferably composed according to the formula MgLi4Al4SE2 mass % (=4 mass % Li+4 mass % Al+2 mass % RE+rest base element Mg), wherein RE is a rare earth metal. Alternatively, the magnesium alloy may also be composed according to the formula MgY4RE3Li2.4 mass % wherein RE is also a rare earth metal. The rare earth metal, e.g. Cerium, improves the mechanical and corrosive properties by removing the hydrogen and producing more surface layer components.

The magnesium alloy may be formed into an implant through molding metallurgy, powder metallurgy or through mechanical alloying, or be applied onto prefabricated implants using metal injection/sinter techniques. The materials may be used as implants in a cast or thermo mechanically treated state. The mechanical and/or corrosive properties are enhanced through sequential extrusion, homogenisation and hardening. The implants can also be produced through machining or shaping methods such as e.g. turning on a lathe, forging or punching.

The invention utilizes the rare earth metals which, as a group, have highly similar mechanical and corrosive properties, which they bring to the alloy. In this case, the alloy components Cerium (representing the class of Cerium-based mixed metals) and Yttrium are used as examples, since these are, at present, the most economical. All other rare earth elements function in a comparable fashion. The rare earths form hydroxides during corrosion, e.g. Ce(OH)₃, aluminum forms spinels such as MgAl₂O₄, and magnesium forms a MgO and Mg(OH)₂ surface layer. With the increased pH of the double layer, the addition of lithium renders these surface layer components thermodynamically more stable and further surface layer components such as e.g. AL(OH)₃ or CeAlO₃ become thermodynamically possible and stable. Enrichment of the cover layer with more components produces a density increase which reduces the intrinsic tension of the Mg(OH)₂ surface layer and reduces diffusion of Mg. Less Mg in the double layer reduces hydrogen production and corrosion of the implant. The reduced amount of hydrogen renders the implant more compatible with the body and the pH value remains at a higher level. The partially pH dependent surface layer components thereby remain intact and reduce the corrosion rate.

The inventive magnesium alloy may be used in the form of surgical mounting wires of different thickness, which may also be woven from individual wires, for screws, in particular for hand and foot surgery and in the traumatological and orthopaedic bone and joint surgery, in particular as interference screws (crucial ligament surgery), and as a suture and anchoring system for fixing muscles, tendons, meniscus, joints (e.g. acetabulum, glenoid), fascies, periost and bondes. The magnesium alloy can also be used for plates, pins, buttons or cerc layers.

In one further possible application, wound or fracture fabric or wound or fracture foils can be produced from the inventive magnesium alloy. They can be produced through mutually connecting thin wires or punching out thin metal sheet.

Moreover, surgical stitching material, in particular wound clips, e.g. for clipping devices, may be made from the magnesium alloy.

Implants with an implant coating comprising the inventive magnesium alloy can be used, in particular, for implants which are in contact with bones. The coating can be applied by conventional methods, e.g. thermal injection (arc and plasma), PVD (physical vapor deposition), CVD (chemical vapor deposition) or co-extrusion.

To increase compatibility in the human or animal body, the inventive magnesium alloy contains no cadmium, i.e. it is cadmium-free.

The use of the inventive magnesium alloy for prostheses or prosthesis parts is advantageous, since the implants can be resorbed after the bone has healed to secondary stability, wherein the natural load distribution within the bone is not impeded.

The effects of the individual alloy components are described below for alloys of the type MgYRE and MgLiAlRE, wherein RE, the rare earth, is preferably Ce. The RE component is illustrated below with Ce.

Al, aluminium: Al additions retard corrosion in an outdoor environment and also in electrolytes. When exposed to various weather conditions, Al-alloyed Mg produces smaller surface layer thicknesses than Mg—Mn or Mg—Fe. Lower oxidation rates may be accompanied by relatively dense surface layers and therefore increased corrosion resistance. Due to the high solubility of 11.8 at %, the structure can be greatly modified in dependence on the solidification rate. High cooling rates generate homogeneous structures with increased corrosion resistance through reduced liquidation, grain refinement and fewer localized elements. Lower cooling rates produce a heterogeneous structure with coarser precipitates. Heterogeneous structures and therefore local micro elements should generally be avoided. Mg(OH)₂, 3Mg(CO)₃.Mg(OH)₂.3H₂O, Cl⁻, CO₃², O²⁻, OH⁻, and Al³⁺ ions have been detected on MgAl3.5 mass % and MgAl10 mass % alloys in a Mg(OH)₂ saturated 3% NaCl solution, wherein the Al³⁺ ion concentration increases with corrosion time. Al³⁺ ions increase surface layer formation not only through forming the spinel MgAl₂O₄ magnesium aluminate, but also since the trivalent cation binds the above-mentioned anions in the surface layer via charge transfer. The Al-rich precipitates thereby act as corrosion barriers having increased corrosion resistance, since the aluminum-rich surface regions generate mixed oxides. MgAl9Zn1 (AZ91) in a 5% NaCl solution in the cast state has a corrosion resistance which is reduced by a factor of 3 compared to the homogenized, uniformly enriched structure. Eutectic Mg—Al precipitates are formed through hardening and the corrosion resistance is doubled. With increasing Al content, the surface layer thickness decreases, since enrichment with Al cations reduces the Mg solution which generates Mg(OH)₂ formation in the surface layer. One can assume that for concentrations above 4 mass % Al, the structure and stability of the corrosion-protecting Al₂O₃ no longer changes: it is integrated in MgO in the form of MgO.Al₂O₃. However, MgO.Al₂O₃ cannot be equated with the stoichiometrically identical MgAl₂O₄. MgO.Al₂O₃ has the elementary cell structure (B1·(D5₁, D5₆, H1₁)≠the MgAl₂O₄ elementary cell structure (H1₁). With increasing Al content in the Mg alloy, the Al₂O₃ content in the surface layer and therefore the corrosion resistance increases. The Al additions reduce intermetallic corrosion. The corrosion-enhancing affinity between Al and Fe, which reduces the Fe limit value and forms cathodic Fe₃Al with E⁰_{5-NaCl}=−0.498 mV can be compensated for through Mn additions, for both sand and pressure die casting. High Al content shifts the maximum limit values for AZ91 (MgAl9Zn1) from pressure die casting to 50 ppm for Fe, 15 ppm for Ni, and 300 ppm for Cu. Magnesium, the most important alloy component, can generally be regarded as corrosion-protecting in amounts of 1 . . . 9 mass %.

Li, lithium: Studies of the Mg—Li system go back to 1910. In the second period of magnesium development, MgLi14 mass % base systems (MgLi38 at %) having high lithium content were examined. They are characterized by a density of 1.4 g/cm³ and by a high reshaping capacity due to the structure which is cubically centered in space at 30 at %, which facilitates sheet metal production e.g. using the alloy MgLi12Al1 mass % (LA141). This extremely light material must be protected during use and the corrosion behavior is chemically and electrochemically unsatisfactory due to the Li alloy component such that the overall performance is generally evaluated as poor. Samples of this material LAE141 in the cast state already tarnish after a short time of four weeks and chipping-off occurs after approximately 6 months (plate-shaped segments break away from the bulk). However, Li additions of up to 10 mass % reduce corrosion in a 5% NaCl solution. In boiling water or in water vapor, the corrosion resistance is inversely proportional to the Li content. Air moisture plays an important role in corrosion of MgLi14 mass % whose corrosion resistance can be increased in this case by 1 mass % Al. For outdoor conditions in an urban, continental sea climate, MgLi40Al3Zn0.3 at %, homogenized with (400° C./30 min/oil) shows the least corrosion, coming quite close to that of AM20: The grooves from machining are still visible after three months and the surface shines weakly and black. After 12 months, the surface of the homogenized MgLi40Al3Zn0.3 at % is destroyed. For Mg material containing Li and more Al, the AlLi phase, refined by 700 mV, whose volume portion is reduced through homogenisation, becomes critical with regard to corrosion. In synthetic sea water, MgLi40Ca0.8 in the cast state shows the least electrochemical corrosion rates. Chemically and electrochemically alkalising Li shifts the pH value in the double layer to pH>11.5, which is in the stability region of Mg(OH)₂. The Mg(OH)₂ surface layer is extended in exemplary systems on the basis of MgLi40 at % by alloy components of Al, Zn or Ca. In addition to the basic increase in the corrosion resistance through mixed oxides, systems such as MgLi40Al3Zn1 at % or MgLi40Ca0.1 at % in 0.01 M H₂SO₄ solution or MgLi40Al3Zn1 at % in tap water containing CO₂ may have a higher corrosion resistance than AZ91 (MgAl9Zn1).

The Li alloy component increases the ductility and accelerates corrosion. In particular, in combination with the alloy component Al, Mg—Li—Al systems cannot be used without protection due to formation of the highly corrosion-promoting AlLi phase. Their use remains limited to the military field due to the difficulties associated with corrosion protection.

Rare earths, RE, with Ce as example: The lanthanoides are referred to as rare earths, rare earth metals, or mixed metals. The rare earths include those elements of the periodic table having atomic numbers 57 . . . 71 plus So and Y which, however, are distinguished in the ASTM nomenclature and discussion due to their differing characteristics for alloys. The oxides are called rare earths (RE or HRE). They are categorized as a group due to their highly similar chemical and metallurgical properties, properties which are transferred to their alloys.

The free corrosion potentials of the rare earth metals are close to Mg such that alloying of Mg must not be regarded as critical right from the outset. Cerium additions, however, reduce the corrosion resistance. For Mg—Al systems, Ce additions, Which are visible on the surface as a light blue to violet glaze, increase the corrosion resistance independent of whether Ce is present in an Al—Ce precipitate (see Al) or is homogenized (410° C./16 h/Water). The three-layer surface topography of the Mg—Al system is Al-enriched and dehydrated through addition of Ce thereby increasing the resistance to the passage of cations. The minimum limit value for corrosion-protecting Al content is reduced by rare earths, in the present case Ce. Ce is the dominating component of the Ce-based RE: 50 mass % Ce, 25 mass % La, 20 mass % Nd and 3 mass % Pr. For Mg alloys which corrode in Mg(OH)₂ saturated 5% NaCl solution, the corrosion rate is reduced in particular by Nd. Nd belongs to the group of HRE, the rare earths with higher relative atomic mass. A Mg—Y—Nd—Zr alloy has the same corrosion rate as the reference alloy MgAl9Zn1 (AZ91), but has a reduced pitting depth. This phenomenon can be explained by an RE enriched Mg(OH)₂ surface layer. Mg—Gd—Y—Zr also has a good corrosion resistance. Y and Nd are recommended as corrosion-protection in the alloy: MgDy10Nd3Zr0.4 mass % has corrosion properties comparable to MgAl9Zn1 (AZ91D).

Y, Yttrium: Y is a rare earth metal and has corrosion properties similar to those of other RE: For a maximum mixed crystal solubility of 12.5 mass % for Y in Mg, the corrosion rate in river water is raised to twice that of MgZn2 through adding increasing amounts of Y of up to Y<4 mass % to the MgZn2. Beyond these amounts, the corrosion rate increases continuously. For MgZn2Y12, the corrosion rate is higher by a factor of 9. This is attributed to the increasing permeation of Mg—Zn-Mk with MgₓY_{z} phases.

Combinations of the Alloy Systems

The corrosion-protecting influence of lithium in a technical alloy is interesting not only under corrosion-specific viewpoints. Li reduces segregation and increases the corrosion resistance and ductility in Mg—Al-RE-systems (AE) since aluminum has a higher affinity to rare earths than to lithium. In other cases, the addition of Li to Mg—Al-systems forms a corrosion-increasing AlLi phase which would reduce the corrosion resistance due to its cathodic character and also through removal of Al from the mixed crystal. On the other hand, Li can stabilize not only the natural surface layer Mg(OH)₂ but also RE(OH)₃ and REAlO₃ with additional surface layer components, via dynamic alkalisation.

The following embodiments concern AE systems with graded Li content: AE42, LAE242, LAE342, LAE442, and LAE542, wherein 12 at % Li corresponds to 4 mass %, when upwardly rounded. The LAE452 and LAE472 alloys have, as suggested by results of experiments for corrosion-protecting alloys, 4 mass % Li, and, as suggested by the findings in AM and AZ systems regarding structure formation in dependence on the Al content, 7 mass % Al, wherein the limiting value for Al is higher than the generally expected 5 mass % due to the grain refinement caused by RE. Maximation of the Al content is desirable due to surface layer density increases associated with magnesium aluminate.

With an increase in the Li component, the electrochemical corrosion rate of the AE system initially slightly increases from 4 mass % for identical Al content. The macroscopic findings of the corrosion samples after 200 h in synthetic sea water show that the increased corrosion rates of LAE542 and LAE452 are due to increased attack in the vicinity of the neutral fibers in the cast bolt and therefore de-mixing.

The metallic shine is due to the fact that, compared to the Li-free variant having surface layer components of Mg(OH)₂, MgAl₂O₄, Al(OH)₃ (the latter only at average pH values) and Ce(OH)₃, a further component CeAlO₃ is stabilized by the pH value increase caused by the Li concentration. The increased pH value makes the Al(OH)₃ unstable which is desirable since the entire aluminum changes into aluminate. The increase in the Al content from 4 mass % through 5 mass % to 7 mass % drastically increases the precipitation output as expected, and changes the type of precipitates. Moreover, the electrochemical corrosion rate is considerably reduced. The highly alloyed LAE472 system is the most stable with respect to corrosion.

The thermo-mechanical modification of LAE472 is effected sequentially in the form of casting, homogenization, extrusion and hardening. In the cast state, Mg—Li-MK is permeated by large surface area colonies of the Al₁₁Ce₃ phase. Homogenization should not only reduce precipitations and intrinsic tensions associated with case-hardening casting, but also fix defined homogenisation and hardening states. Homogenization is carried out for the sample materials with (350° C./4 h/oil), wherein the semi-finished products are wrapped in a thermal treatment foil which reduces vaporization and reduces diffusion of lithium. In contrast to LAE442, the structure of LAE472 cannot be completely homogenized and freed from local micro elements due to the strong precipitation. Thermal treatment does not draw a large portion of the precipitates into the matrix rather causes considerable coarsening and increased precipitates in the structure in consequence of coagulation. In accordance with the principles of the corrosion-protecting alloying, the corrosion resistance of the homogenized LAE472 vs. LAE442 is reduced in the cast state.

The thermo-mechanical treatment consists of two steps: 30 minutes preheating at 350° C. and subsequent full power forward extrusion at 300° C.

Extrusion produces new precipitates in the structure which again influence the mechanical properties and the corrosion resistance. Renewed hardening (180° C./16 h/oil) provides uniform distribution of the precipitates. The precipitate portion of the global Al₂Ce and laminar Al₄Ce phases remains unchanged; however the grain size is reduced. The corrosion rate of the LAE472 is at a maximum in the cast state with 0.04 mm/a in a sample run, however, considerably below the order of magnitude of the initial material MgAl4SE2 mass % with 0.2 mm/a. The corrosion rates of the thermally treated materials suggest a different model for corrosion-protecting alloying. The poorly homogenized LAE72, having large amounts of localized elements, does not have the lowest corrosion rate, rather the extruded LAE472 which is permeated with fine local cathodes during hardening to yield 0.025 mm/a. A further principle of corrosion-protecting alloying is therefore the defined permeation of the matrix with more refined phases, so-called local cathodes, to produce an increase in the corrosion resistance compared to the cast state.

For electro-chemical corrosion measurements in the aggressive synthetic sea water medium, all LAE472 systems have extremely low corrosion rates but are not kinetically stabilized: The corrosion rates increase with time. LAE systems with a maximum of 4 mass % Al do not exhibit this behavior over an examination duration of 200 h. This difference permits classification into accelerated and “normally” corroding implant materials, since the corrosion rates shown herein are proportionally higher in vivo.

Surface layer enhancement caused by the rare earths, Ce in the present case, suggests empirical examination of the corrosion resistance of Mg—Y-RE- (WE) alloys in-vivo implant material. The results confirm the corrosion-inhibiting effect of Ce, wherein these alloys have a higher stability and lower ductility. Due to the uncritical constitution of the two-material-systems, one can conclude through metallurgical and metal-physical rules that further increases in the rare earth portion will lead to further increases in the corrosion resistance.

1. through 6. Cancelled.

7. A medical implant for the body of a person or an animal made at least partially from a magnesium alloy, the magnesium alloy comprising:
0.01 to 7 mass % lithium;
0.01 to 8 mass % rare earth metal;
0.01 to 7 mass % yttrium; and
the rest magnesium and impurities.

8. The medical implant according to claim 1, further comprising 0.01 to 16 mass % aluminum.

9. The medical implant of claim 1, wherein said rare earth metal comprises at least one of cerium, neodymium, and praseodymium.

10. The medical implant of claim 2, wherein the magnesium alloy is composed according to the formula MgLi4Al4RE2 mass %, wherein RE is a rare earth metal.

11. The medical implant of claim 1, wherein the magnesium alloy is composed according to the formula MgY4RE3Li2.4 mass %, wherein RE is a rare earth metal.
